(19) United States
(12) Patent Application Publication
(10) Pub. No.: US 2026/0272966 A1
(43) Pub. Date: Sep. 17, 2026

(54) COMBINATION TREATMENT FOR OVARIAN CANCER

(71) Applicant: THE JOHNS HOPKINS UNIVERSITY, Baltimore, MD (US)

(72) Inventors: Robert A. CASERO, Baltimore, MD (US); Tracy Murray STEWART, Baltimore, MD (US); Cassandra HOLBERT, Baltimore, MD (US)

(73) Assignee: THE JOHNS HOPKINS UNIVERSITY, Baltimore, MD (US)

(21) Appl. No.: 19/472,593

(22) PCT Filed: Apr. 3, 2024
(86) PCT No.: PCT/US2024/022817

§ 371 Date: Oct. 3, 2025

Related U.S. Application Data
(60) Provisional application No. 63/493,907, filed on Apr. 3, 2023.

(51) Int. Cl. A61K 31/704; A61K 31/198; A61K 31/7068; A61K 45/06; A61P 35/00

(52) U.S. Cl. CPC A61K 31/704; A61K 31/198; A61K 31/7068; A61K 45/06; A61P 35/00

(57) ABSTRACT

Provided are methods for treating patients having ovarian cancer. The methods comprise administering a combined effective amount of SBP-101 and either doxorubicin or eflornithine.

### REFERENCE TO RELATED APPLICATIONS

The present application claims the priority benefit of U.S. provisional application No. 63/493,907, filed Apr. 3, 2023, the entire contents of which are incorporated herein by reference.

### STATEMENT OF FEDERALLY SPONSORED RESEARCH

This invention was made with government support under Grant No. R01CA235963 awarded by the National Institutes of Health. The government has certain rights in the invention.

### BACKGROUND

### 1. Field

The present disclosure relates generally to the field of cancer. More particularly, it concerns compositions and methods useful for treating ovarian cancer.

### 2. Description of Related Art

Ovarian cancer was the third most commonly diagnosed gynecological cancer in 2020 and is predicted to rise in incidence over 40% by 2040 (Sung et al., 2021). As over half of patients already have metastatic disease at diagnosis, ovarian cancer is the leading cause of gynecological cancer deaths, with a five-year relative survival for patients diagnosed with distant disease of approximately 30% (Cronin et al., 2022). Standard therapy for advanced ovarian cancer typically consists of surgical debulking and a chemotherapy regimen. Initial chemotherapy consists of either taxane or platinum chemotherapy, or a combination thereof. While approximately 75% of patients respond to front line therapy, 70% of those patients who initially respond eventually relapse within 1 to 3 years. After relapse, patients respond moderately or poorly to subsequent chemotherapy. Additionally, intolerance to platinum agents is a clinical concern, as the risk of cumulative toxicities increases over the course of continued treatments (Muñoz-Galván & Carnero, 2020; de Costa & Baiocchi, 2021; Ghoneum et al., 2021). There is a significant unmet need due to the high recurrence rate, despite an initially high response rate. As such, the discovery of new therapeutic options and combinations are essential to overcome the unmet therapeutic needs of the platinum-resistant patient population.

### SUMMARY

Provided herein are combinations of ivospemin treatment with chemotherapies commonly used in the treatment of platinum-resistant ovarian cancer.

Provided herein are methods of treating a patient having an ovarian cancer, the methods comprising administering to the patient a combined effective amount of (i) SBP-101 and (ii) doxorubicin, gemcitabine, or topotecan. The (i) SBP-101 and (ii) doxorubicin, gemcitabine, or topotecan may be administered consecutively. The SBP-101 may be administered prior to the doxorubicin, gemcitabine, or topotecan. The SBP-101 may be administered after the doxorubicin, gemcitabine, or topotecan. The (i) SBP-101 and (ii) doxorubicin, gemcitabine, or topotecan may be administered simultaneously. The (i) SBP-101 and (ii) doxorubicin, gemcitabine, or topotecan may be co-formulated.

Provided herein are pharmaceutical compositions comprising (i) SBP-101 and (ii) doxorubicin, gemcitabine, or topotecan, and a pharmaceutically acceptable carrier. The compositions may be for use in the treatment of ovarian cancer. Provided herein are pharmaceutical compositions comprising SBP-101, for use in the treatment of ovarian cancer in combination with a second pharmaceutical therapy comprising doxorubicin, gemcitabine, or topotecan. Provided herein are pharmaceutical compositions comprising doxorubicin, gemcitabine, or topotecan, for use in the treatment of ovarian cancer in combination with a second pharmaceutical therapy comprising SBP-101. Provided herein are kits comprising a first pharmaceutical composition comprising SBP-101 and a second pharmaceutical composition comprising doxorubicin, gemcitabine, or topotecan.

Provided herein are methods of treating a patient having an ovarian cancer, the methods comprising administering to the patient a combined effective amount of SBP-101 and an ornithine decarboxylase (ODC) inhibitor. The ornithine decarboxylase (ODC) inhibitor may be DFMO. The SBP-101 and the ODC inhibitor may be administered consecutively. The SBP-101 may be administered prior to the ODC inhibitor. The SBP-101 may be administered after the ODC inhibitor. The SBP-101 and the ODC inhibitor may be administered simultaneously. The SBP-101 and the ODC inhibitor may be co-formulated.

Provided herein are pharmaceutical compositions comprising SBP-101, an ODC inhibitor, and a pharmaceutically acceptable carrier. The compositions may be for use in the treatment of ovarian cancer. Provided herein are pharmaceutical compositions comprising SBP-101, for use in the treatment of ovarian cancer in combination with a second pharmaceutical therapy comprising an ODC inhibitor. Provide herein are pharmaceutical compositions comprising an ODC inhibitor, for use in the treatment of ovarian cancer in combination with a second pharmaceutical therapy comprising SBP-101. Provided herein are kits comprising a first pharmaceutical composition comprising SBP-101 and a second pharmaceutical composition comprising an ODC inhibitor.

The ovarian cancer in the subject may have been previously treated with the anti-cancer agent or at least one other anti-cancer agent. The ovarian cancer may have developed resistance to a platinum-based therapy. The ovarian cancer may be a platinum-resistant ovarian cancer.

The methods or use may further comprise administering at least one additional therapeutic to the subject. The at least one additional therapeutic may be selected from the group consisting of surgical therapy, chemotherapy, radiation therapy, cryotherapy, hormonal therapy, toxin therapy, immunotherapy, or cytokine therapy.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Chemotherapy combination study in vivo. VDID8⁺ murine ovarian cancer model was administered SBP-101 in combination with doxorubicin (dosage of 1 mg/kg MWF for 4 weeks=12 total doses≈approximately 250 μg total).

FIG. 2. Treatment of A2780 ovarian cells with SBP-101 in combination with doxorubicin.

FIG. 3. Treatment of A2780 ovarian cells with SBP-101 in combination with DFMO.

FIGS. 4A-4B. Ivospemin treatment reduces cell viability in ovarian adenocarcinoma cells in vitro regardless of cisplatin sensitivity. Ovarian adenocarcinoma cell lines were treated for 96 hours with increasing concentrations of ivospemin ranging from 500 nM to 10 μM. Ivospemin treatment decreased cell viability in both cisplatin-sensitive (FIG. 4A) and cisplatin-resistant (FIG. 4B) cell lines. IC₅₀ values are listed in Table 1.

FIGS. 5A-5D. Ivospemin increases the toxicity of gemcitabine and topotecan in ovarian adenocarcinoma cell lines. Four ovarian adenocarcinoma cell lines were treated with 2 μM ivospemin for 96 hours, with the inclusion of chemotherapeutic agents for the last 24 hours at the following concentrations: gemcitabine: 50 nM; topotecan: 50 nM; paclitaxel: 2 nM; docetaxel 2 nM. Ivospemin increased the toxicity of all four chemotherapeutics in both A2780 and ACRP cell lines (FIGS. 5A, 5B), but only combinations with gemcitabine and topotecan significantly exceeded the effect of ivospemin alone. Ivospemin similarly increased the toxicity of gemcitabine and topotecan in the cisplatin-sensitive cell line OV90 (FIG. 5C) and cisplatin-resistant cell line CaOV-3 (FIG. 5D).

FIG. 6. Ivospemin increases survival over chemotherapy alone in the VDID8+ model of ovarian cancer. Female C57Bl/6J mice were injected with 350,000 VEGF+, Defensin+ID8 cells per mouse. Treatment with all three drugs began on day 3 post-injection at the following doses: ivospemin (24 mg/kg 2qw×3, alternating weeks); gemcitabine (30 mg/kg qw×4); topotecan (1 mg/kg 3qw×4). Ivospemin monotherapy increased median survival from 43 days to 52 days post-injection. Both combination therapies increased median survival to approximately 62 days post-injection (~45% increase).

FIGS. 7A-7C. Ivospemin improves the survival benefit of chemotherapy by delaying disease onset and decreasing tumor burden. While ivospemin alone did not influence time to ascites formation, addition of ivospemin to either gemcitabine or topotecan monotherapy increased the number of days between cell injection and ascites formation (FIG. 7A). Ivospemin treatment decreased ascites volume at the first drain (FIG. 7B) either as a monotherapy or in combination with chemotherapy. Chemotherapy had no effect on ascites volume. This decreased ascites volume following ivospemin treatment was sustained through the second drain (FIG. 7C), resulting in reduced tumor burden of approximately 50%.

FIG. 8. Ivospemin improves survival benefit of doxorubicin even at subclinical dosing levels. Female C57Bl/6J mice were injected with 350,000 VEGF+ Defensin+Id8 cells per mouse. Treatment with both drugs began 3 days post-injection at the following doses: ivospemin (24 mg/kg 2qw×4, alternating weeks); doxorubicin (0.5 mg/kg 2qw×4). The addition of ivospemin to low-dose doxorubicin improved median survival from 96 to 114 days, a 20% increase in survival (p-value 0.03). The combination treatment results in a 128% increase in median survival over untreated animals with a p-value=<0.0001.

FIG. 9. Ivospemin improves survival benefit of subclinical doxorubicin by delaying tumor onset and decreasing overall tumor burden. Mice co-treated with ivospemin (24 mg/kg 2qw×4 alt week) and subclinical doxorubicin (0.5 mg/kg 2qw×4) benefited from an approximate 16.8-day delay in ascites onset (approximately 20%) compared to doxorubicin single agent animals with a p-value of 0.04. The addition of ivospemin to low dose doxorubicin also decreased overall tumor burden as measured by ascites. Combination treated mice exhibited a 50% reduction in ascites volume at first drain (p-value of 0.019) and a 62% reduction in ascites volume at second drain (p-value of 0.007).

FIG. 10. Ivospemin and doxorubicin cooperatively decrease polyamine content of ascites fluid in treated mice. Ascites fluid was collected from the second drain of animals treated with ivospemin and low-dose doxorubicin. Red blood cells were lysed and the remaining cells (including tumor, immune and stromal) were pelleted and acid extracted for quantification of polyamine content by HPLC. Combination treated animals had the lowest ascitic polyamine content, exhibiting a 73% reduction in total polyamine content to mock (p-value=0.0016) and a 40% reduction compared to doxorubicin alone (p-value=0.02). Ivospemin could only be detected by HPLC in the ascites of animals that had been treated with ivospemin. N1-acetlyated spermidine could be detected in doxorubicin and combination ascites, though at a very low level. This indicates a possible upregulation of polyamine catabolism.

FIG. 11. Ivospemin and doxorubicin cooperatively upregulate polyamine catabolism to reduce cellular polyamine content. The human ovarian adenocarcinoma line was treated with ivospemin (5 μM) and doxorubicin (500 nM) for 48 hours. The activity of the polyamine catabolic enzyme SSAT and the polyamine biosynthetic enzyme ODC was evaluated. Either ivospemin or doxorubicin treatment only increases SSAT activity with the combination treatment having the highest catabolic activity (approximately double either single agent). Ivospemin alone or in combination with doxorubicin decreases polyamine biosynthesis through depression of ODC activity. Doxorubicin alone does not have any effect on ODC activity.

FIGS. 12A-12B. Efficacy of the combination of ivospemin and doxorobucin is dependent on an intact immune system. Immunocompromised NSG mice were injected with 250,000 VDID8+ cells per mouse and subsequently treated with 24 mg/kg 2qw×4 alternating weeks ivospemin and a subclinical dose of doxorubicin (0.5 mg/kg 2qw×4). Even at a subclinical dosing level, doxorubicin alone produced some toxicities among treated animals, most notably weight loss and anemia, however none of the three treatment arms result in any statistically significant survival benefit (FIG. 12A). Neither ivospemin alone nor the combination treatment decrease tumor burden or delayed ascites formation as previously seen in immunocompetent models (FIG. 12B).

### DETAILED DESCRIPTION

Polyamines are small cationic alkylamines that play critical roles in many essential cell processes including cellular growth, proliferation, and apoptosis. Due to their positive charge at physiological pH, polyamines interact with negatively charged macromolecules to influence critical cellular processes (Pegg, 2009). As such, cancers are fully reliant on increased polyamine pools that are maintained through dysregulation of tightly controlled polyamine metabolism. Pharmaceutical modulation of polyamine metabolism is a promising avenue in cancer therapeutics and has been attempted with numerous enzyme inhibitors and polyamine analogues. As such, improved methods of using polyamine metabolism modulators and polyamine analogues are provided herein.

### I. DEFINITIONS

As used herein, the term “subject” or “patient” refers to any organism to which a composition in accordance with the present disclosure may be administered, e.g., for experimental, diagnostic, prophylactic, and/or therapeutic purposes. Typical subjects include animals (e.g., mammals such as mice, rats, rabbits, non-human primates, and humans) and/or plants. Preferably “patient” refers to a human subject who may seek treatment, is in need of treatment, is receiving treatment, will receive treatment, or a subject who is under care by a trained professional for a particular disease or condition.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable excipient” refers to a diluent, adjuvant, excipient or carrier with which a compound of the disclosure is administered. A pharmaceutically acceptable excipient is generally a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include water, any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this present disclosure.

The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other.

The term “chemotherapeutic agent” refers to a compound or a derivative thereof that can interact with a cancer cell, thereby reducing the proliferative status of the cell and/or killing the cell, for example, by impairing cell division or DNA synthesis, or by damaging DNA, effectively targeting fast dividing cells.

The phrase “therapeutically effective amount” or an “effective amount” refers to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring pharmacodynamic effects, and it can be adjusted in connection with the dosing regimen and diagnostic analysis of the subject's condition, and the like. In reference to cancer or pathologies related to unregulated cell division, a therapeutically effective amount refers to that amount which has the effect of (1) reducing the size of a tumor (i.e. tumor regression), (2) inhibiting (that is, slowing to some extent, preferably stopping) aberrant cell division, for example cancer cell division, (3) preventing or reducing the metastasis of cancer cells, and/or, (4) relieving to some extent (or, preferably, eliminating) one or more symptoms associated with a pathology related to or caused in part by unregulated or aberrant cellular division, including for example, cancer.

As used herein any form of administration or coadministration of a “combination”, “combined therapy” and/or “combined treatment regimen” refers to at least two therapeutically active drugs or compositions which may be administered or co-administered, simultaneously, in either separate or combined formulations, or sequentially at different times separated by minutes, hours or days, but in some way act together to provide the desired therapeutic response.

The terms “co-administration” and “in combination with” include the administration of two or more therapeutic agents simultaneously, concurrently, or sequentially within no specific time limits unless otherwise indicated. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., without limitation, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., without limitation, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, the term “parenteral” refers to dosage forms that are intended for administration as an injection or infusion and includes subcutaneous, intravenous, intra-arterial, intraperitoneal, intracardiac, intrathecal, and intramuscular injection, as well as infusion injections usually by the intravenous route.

The terms “treating” or “treatment” of a disease (or a condition or a disorder) as used herein refer to preventing the disease from occurring in a human subject or an animal subject that may be predisposed to the disease but does not yet experience or exhibit symptoms of the disease (prophylactic treatment), inhibiting the disease (slowing or arresting its development), providing relief from the symptoms or side-effects of the disease (including palliative treatment), and causing regression of the disease. With regard to cancer, these terms also mean that the life expectancy of an individual affected with a cancer may be increased or that one or more of the symptoms of the disease will be reduced. “Treating” also includes enhancing or prolonging an anti-tumor response in a subject.

“Progression free survival (PFS),” as used in the context of the cancers described herein, refers to the length of time during and after treatment of the cancer until objective tumor progression or death of the patient. The treatment may be assessed by objective or subjective parameters; including the results of a physical examination, neurological examination, or psychiatric evaluation. In preferred aspects, PFS may be assessed by blinded imaging central review and may further optionally be confirmed by ORR or by blinded independent central review (BICR).

“Overall survival (OS)” may be assessed by OS rate at certain time points (e.g., 1 year and 2 years) by the Kaplan-Meier method, and corresponding 95% CI will be derived based on the Greenwood formula by study treatment for each tumor type. OS rate is defined as the proportion of participants who are alive at the time point. OS for a participant is defined as the time from the first dosing date to the date of death due to any cause.

As used herein a “complete response” is the disappearance of all signs of cancer in response to treatment. A complete response may also be referred to herein as “total remission”.

As used herein the term “partial response” means a decrease in the size of the tumor, or in the extent of cancer in the body in response to treatment. A partial response may also be referred to herein as “partial remission”.

The term “cancer”, as used herein, shall be given its ordinary meaning, as a general term for diseases in which abnormal cells divide without control.

The term “reducing a tumor” or “tumor regression” as used herein refers to a reduction in the weight, size or volume of a tumor mass, a decrease in the number of metastasized tumors in a subject, a decrease in the proliferative status (the degree to which the cancer cells are multiplying) of the cancer cells. For example, the weight, size or volume of a tumor may be reduced by about 1%, 5%, 10%, 15%, 20%, 25%, 30%, 35%, 40%, 45%, 50%, 55%, 60%, 65%, 70%, 75%, 80%, 85%, 90%, 95% or 98% or more as compared to baseline. Techniques for establishing whether a tumor has been reduced or regressed are known in the art.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

### II. SBP-101

Polyamines (PA) including spermine are ubiquitous biological molecules found in all mammalian cells. Polyamines are essential for the growth, reproduction and function of normal cells, and programmed cell death (apoptosis). Each of the three native polyamines (spermine, spermidine and putrescine) are metabolized intracellularly with levels maintained within narrow ranges by a series of enzymes including ornithine decarboxylase (ODC), S-adenosylmethione decarboxylase (SAMDC/AMD1), spermidine/spermine N1-acetyltransferase (SSAT), spermine oxidase (SMOX), acetylpolyamine oxidase (PAOX) and others. Polyamine catabolism via SMOX or SSAT and PAOX generates hydrogen peroxide (H₂O₂) and aldehydes, which can further induce SMOX and SSAT leading to apoptosis, and may, if unchecked, lead to a positive cell-death-signal-generating cycle that can be therapeutically beneficial when isolated to the cancer cells.

Increased biosynthesis of polyamines and their biosynthetic enzymes in neoplastic tissues has made this class of molecules a promising target for cancer therapeutic efforts. The polyamine transport uptake mechanism appears to be up regulated in various tumor types, including pancreatic ductal adenocarcinoma where demand for polyamines is high.

Inducing polyamine depletion via the cellular uptake of dysfunctional synthetic polyamine analogues has been proposed as an antitumor strategy. Polyamine analogues are an encouraging therapeutic strategy aimed at exploiting the tight self-regulation of polyamine homeostasis in neoplastic cells. A promising class of these analogues involve the alkylation of the primary amine groups of spermine, the largest of the naturally occurring mammalian polyamines (Bergeron et al., 2000; Bergeron et al., 1988; Casero & Marton, 2007; Casero & Woster, 2001). The traditional mechanism of action for polyamine analogues is to decrease intracellular polyamine levels through feedback inhibition. These compounds compete with endogenous polyamines for uptake, and upon intracellular accumulation, stimulate catabolism of higher order polyamines and reduce polyamine biosynthesis (Casero & Woster, 2001; Fogel-Petrovic et al., 1996; Fogel-Petrovic et al., 1996; Pegg & Hu, 1995). By being sufficiently dissimilar to the natural polyamines, therapeutically relevant polyamine analogues are unable to support critical cellular functions, thereby starving cancer cells of necessary polyamines.

Polyamine analogues enter cells via polyamine transporters, substitute for natural polyamines in their self-regulatory roles, but fail to function as natural polyamines in promoting cell growth. Consequently, a state of “pseudo-polyamine” excess is created in cells, thereby downregulating the enzymes responsible for polyamine synthesis, and in some cases inducing SSAT and/or SMOX, key enzymes responsible for intracellular polyamine catabolism.

Several polyamine analogues, including bis(ethyl) norspermine (BENSpm/DENSpm) and diethylhomospermine (DEHSPM), have demonstrated efficacy against cancer both in vitro and in vivo. However, they elicited notable off-target effects and toxicities in early clinical trials (Hahm et al., 2002; Creaven et al., 1997; Wilding et al., 2004; Murray Stewart et al., 2020a; Murray Stewart et al., 2020b). Although it is likely that these off-target effects were a result of the dosing schemas and not the analogues themselves, the field has progressed by designing less-toxic derivatives of the first-generation polyamine analogues and improving dosing regimes (Bergeron et al., 2000).

SBP-101 is a small-molecule, ethylated and hydroxylated derivative of homospermine, a dysfunctional analogue of the naturally occurring polyamine spermine and has the following Formula 1.

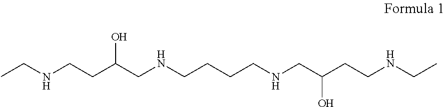 [SMILES: CCNCCC(O)CNCCCCNCC(O)CCNCC]

SBP-101 (also known as diethyl dihydroxyhomospermine; (HO)2DEHSPM; 3,8,13,18-tetraazaicosane-6,15-diol) is a spermine analogue that has shown efficacy in slowing pancreatic and ovarian tumor progression both in vitro and in vivo. In addition, this compound is of use in the resection of the exocrine pancreas and in the treatment of pancreatitis and other diseases and disorders of the pancreas (see, for example, U.S. Pat. No. 6,160,022 and WO 2017/062704).

Ivospemin (SBP-101) is a hydroxylated derivative of the spermine analogue DEHSPM. Previous studies have shown that ivospemin inhibits pancreatic and ovarian cancer both in vitro and in vivo (Holbert et al., 2022; Shah et al., 2014). Results from a multicenter Phase 1a/b trial suggest that ivospemin is a tolerable and potentially advantageous addition to standard of care (gemcitabine and nab-paclitaxel) in previously untreated metastatic pancreatic ductal adenocarcinoma (PDA) patients (Singhal et al., 2021; Therapeutics, 2022). The combination of ivospemin and gemcitabine/nab-paclitaxel in metastatic PDA has progressed to the multi-center ASPIRE Phase 2/3 clinical trial (NCT05254171) that is currently recruiting patients (Therapeutics, 2023). Previous studies have shown that ivospemin decreases the viability of lung, pancreatic, and ovarian adenocarcinoma cell lines in vitro through modulation of enzymes within the polyamine metabolic pathways (Holbert et al., 2022). Ivospemin treatment of a syngeneic ovarian cancer murine model significantly decreased tumor burden and increased median survival (Holbert et al., 2022).

It will be appreciated by those skilled in the art that the compound of Formula 1 contains at least two chiral centers. The compound of Formula 1 may exist in the form of two different optical isomers (i.e., (+) or (−) enantiomers) and a diastereomer. All such enantiomers, diastereomers and mixtures thereof including racemic mixtures are included within the scope of the disclosure. The enantiomers of the compound of Formula 1 can be obtained by methods disclosed in U.S. Pat. No. 6,160,022 and WO 2019/152323. The enantiomers of the compound of Formula 1 can also be obtained from a racemic mixture by methods well known in the art, such as chiral HPLC and chemical resolution. Alternatively, the enantiomers of the compound of Formula 1 can be synthesized by using optically active starting materials.

(S,S)—(HO)2DEHSPM is the S,S enantiomer of Formula 1. The chemical name for (S,S)—(HO)2DEHSPM is (6S,15S)-3,8,13,18-teraazaicosane-6,15-diol or N¹, N¹⁴-diethyl-3S, 12S-dihydroxyhomospermine and may also be referred to hereina (HO)2DEHSPM). The CAS number for (S,S)—(HO)2DEHSPM is 259657-09-5. The compound is preferably isolated, formulated and administered in the form of a pharmaceutically acceptable salt, and all reference to (S,S)—(HO)2DEHSPM in relation to compositions and administration refers to free base and salt forms unless otherwise stated. The preferred form of SBP-101 is the stable tetrahydrochloride salt, referred to herein as (S,S)—(HO)2DEHSPM·4HCl.

SBP-101 or a pharmaceutically acceptable salt thereof, is preferably formulated as a pharmaceutical composition with one or more pharmaceutically acceptable diluents, carriers or excipients. The pharmaceutical compositions are preferably formulated for administration to a patient by injection, preferably, parenteral injection and even more preferably by subcutaneous injection. Preferably SBP-101 is formulated in the form of (S,S)—(HO)2DEHSPM·4HCl, for example, in a clear sterile solution in pH-adjusted sterile water for injection, preferably subcutaneous injection. However, other modes of administration of SBP-101 or a pharmaceutically acceptable salt thereof are also contemplated, such as oral.

One dosing regimen for SBP-101 may comprise, for example, daily dosing for 5 consecutive days (e.g., days 1-5) of each treatment cycle for no more than 5 consecutive treatment cycles, optionally no more than 4, 3, 2, or 1 consecutive treatment cycles, wherein a treatment cycle is 28 days. The amount of each dose of SBP-101 in any treatment regimen of the disclosure may be, but is not limited to, 0.2 mg/kg/day (0.14 mg/kg/day), 0.4 mg/kg/day (0.27 mg/kg/day), and 0.6 mg/kg/day (0.41 mg/kg/day).

Another dosing regimen may comprise a combination of a daily dosing regimen of SBP-101 with periodic dosing of SBP-101 in the same or different treatment cycles. One combination daily/periodic dosing regimen comprises administering a daily dosing regimen of SBP-101 as described above followed by one or more treatment cycles wherein SBP-101 is administered periodically, for example on days 1, 8 and 15 of each of the following treatment cycles wherein each treatment cycle is 28 days. In one embodiment, the patient is treated for two treatment cycles of the daily dosing regimen followed by 4 to 6 treatment cycles of the periodic dosing regimen.

In certain embodiments, a dosing regimen is continued until the patient has received a pre-specified total cumulative dose (“TCD”) of SBP-101. In certain embodiments, the TCD is about 12 mg/kg (about 8.2 mg/kg) or less or about 10 mg/kg (about 6.9 mg/kg) or less. In certain embodiments, the TCD is from about 5 mg/kg (about 3.4 mg/kg) to about 12 mg/kg (about 8.2 mg/kg), about 5 mg/kg (about 3.4 mg/kg) to about 10 mg/kg (about 6.9 mg/kg), about 8 mg/kg (about 5.5 mg/kg) to about 10 mg/kg (about 6.9 mg/kg), about 8.5 mg/kg (about 5.8 mg/kg) to about 9.5 mg/kg (about 6.5 mg/kg), about 8.6 mg/kg (about 5.9 mg/kg) to about 9.0 mg/kg (about 6.2 mg/kg), or about 8.8 mg/kg (about 6.0 mg kg).

Another dosing regimen may comprise only periodic dosing of SBP-101 for one or more treatment cycles (i.e., no daily dosing). Periodic dosing only regimens include dosing SBP-101 periodically for no more than about 5 to no more than about 14 doses per treatment cycle wherein dosing occurs on non-consecutive days. One exemplary periodic dosing regimen includes administering SBP-101 on days 1, 8 and 15 of each of any one or more treatment cycles and preferably for at least 1, 2, 5, 8 or more treatment cycles. Another exemplary periodic dosing regimen includes dosing SBP-101 periodically for no more than about 5 to no more than about 10 doses for the first and second treatment cycles wherein dosing occurs on non-consecutive days and thereafter administering SBP-101 on days 1, 8 and 15 of all treatment cycles thereafter (e.g., cycles 3 through 5 or more).

It is understood that any of the dosing schedules of the disclosure may be carried out for 1 or more treatment cycles, such as 1 or more 28-day treatment cycles. Preferably a patient is treated for at least 2, preferably at least 3, preferably at least 4, preferably at least 5, preferably at least 6, preferably at least 8, and preferably at least 10 or more treatment cycles, or until a complete or partial response, disease progression or unacceptable toxicity occurs.

### III. DOXORUBICIN

Doxorubicin (trade name Adriamycin®; pegylated liposomal form trade name Doxil®; nonpegylated liposomal form trade name Myocet®), also known as hydroxydaunorubicin and hydroxydaunomycin, is a drug used in cancer chemotherapy and derived by chemical semisynthesis from a bacterial species. It is an anthracycline antibiotic (note: in this context, this does not mean it is used to treat bacterial infections) closely related to the natural product daunomycin and like all anthracyclines, it is believed to work by intercalating DNA, with the most serious adverse effect being life-threatening heart damage. It is commonly used in the treatment of a wide range of cancers, including hematological malignancies (blood cancers, like leukemia and lymphoma), many types of carcinomas (solid tumors) and soft tissue sarcomas. It is often used in combination chemotherapy as a component of various therapeutic regimens. In some embodiments, doxorubicin can be substituted for another anticancer agent that can be disposed within the aqueous interior of the liposome. Doxorubicin (CAS Reg. No. 23214-92-8) is also named as (8S,10S)-10-(4-amino-5-hydroxy-6-methyl-tetrahydro-2H-pyran-2-yloxy)-6,8,11-trihydroxy-8-(2-hydroxyacetyl)-1-methoxy-7,8,9,10-tetrahydrotetracene-5,12-dione.

Common adverse effects of doxorubicin include hair loss (seen in most of those treated with the drug), myelosuppression (a compromised ability of the body's bone marrow to produce new blood cells), nausea and vomiting (which are seen in roughly 30-90% of people treated with the drug), oral mucositis, oesophagitis, diarrhea, skin reactions (including hand-foot syndrome) and localized swelling and redness along the vein in which the drug is delivered. Less common, yet serious reactions include hypersensitivity reactions (including anaphylaxis), radiation recall, heart damage and liver dysfunction.

The drug is administered intravenously, as the hydrochloride salt. It is sold under a number of different brand names, including Adriamycin® PFS, Adriamycin® RDF, or Rubex®. Doxorubicin is photosensitive, and containers are often covered by an aluminum bag and/or brown wax paper to prevent light from affecting it. Doxorubicin is also available in liposome-encapsulated forms as Doxil® (pegylated form), Myocet® (nonpegylated form), and Caelyx®, although these forms must also be given by intravenous injection.

In various embodiments, methods provided herein can increase the efficacy of and/or decrease undesired side effects from, the doxorubicin.

### IV. EFLORNITHINE

Eflornithine is an enzyme-activated, irreversible inhibitor of ornithine decarboxylase (ODC), the first rate-limiting enzyme of the polyamine biosynthetic pathway. The term “eflornithine” when used by itself and free of context refers to 2,5-diamino-2-(difluoromethyl) pentanoic acid in any of its forms, including non-salt and salt forms (e.g., eflornithine HCl), anhydrous and hydrate forms of non-salt and salt forms (e.g., eflornithine hydrochloride monohydrate), solvates of non-salt and salt forms, its enantiomers (R and S forms, which may also by identified as d and/forms), and mixtures of these enantiomers (e.g., racemic mixture). By “substantially optically pure preparation” is meant a preparation of a first enantiomer which contains about 5% wt. or less of the opposite enantiomer. Specific forms of eflornithine include eflornithine hydrochloride monohydrate (i.e., CAS ID: 96020-91-6; MW: 236.65), eflornithine hydrochloride (i.e., CAS ID: 68278-23-9; MW: 218.63), and anhydrous free base eflornithine (i.e., CAS ID: 70052-12-9; MW: 182.17). Where necessary, the specific form of eflornithine has been further specified. In some embodiments, the eflornithine of the present disclosure is eflornithine hydrochloride monohydrate (i.e., CAS ID: 96020-91-6). The terms “eflornithine” and “DFMO” are used interchangeably herein. DFMO is an abbreviation for difluoromethylornithine. Other synonyms of eflornithine and DFMO include: α-difluoromethylornithine, 2-difluoromethylornithine, 2-(difluoromethyl)-DL-ornithine, 2-(difluoromethyl)-dl-ornithine, 2-(Difluoromethyl) ornithine, DL-α-difluoromethylornithine, N-Difluoromethylornithine, αδ-diamino-α-(difluoromethyl) valeric acid, and 2,5-diamino-2-(difluoromethyl) pentanoic acid.

Eflornithine is relatively non-toxic at low doses to humans while producing inhibition of putrescine synthesis in tumors. Studies in a rat-tumor model demonstrate that eflornithine infusion can produce a 90% decrease in tumor putrescine levels without suppressing peripheral platelet counts. Side effects observed with eflornithine include effects on hearing at high doses, which resolve when it is discontinued. These effects on hearing are not observed at lower doses when administered for up to four year. In addition, a few cases of dizziness/vertigo are seen that resolve when the drug is stopped. Thrombocytopenia has been reported predominantly in studies using high “therapeutic” doses of eflornithine (>1.0 g/m²/day) and primarily in cancer patients who had previously undergone chemotherapy or patients with compromised bone marrow. DFMO treatment can also lead to increased polyamine transport, thus potentially increasing the amount of SBP-101 accumulated by cancer cells.

### V. PHARMACEUTICAL COMPOSITIONS AND FORMULATIONS

The therapeutic compounds of the present disclosure may be administered by a variety of methods, e.g., orally or by injection (e.g., subcutaneous, intravenous, intraperitoneal, etc.) or by perfusion/infusion. Depending on the route of administration, the therapeutic compounds may be coated in a material to protect the compound from the action of acids and other natural conditions that may inactivate the compound.

To administer the therapeutic compound by other than parenteral administration, it may be necessary to coat the compound with, or co-administer the compound with, a material to prevent its inactivation. For example, the therapeutic compound may be administered to a patient in an appropriate carrier, for example, a diluent. Pharmaceutically acceptable diluents include saline and aqueous buffer solutions.

Pharmaceutical compositions suitable for injectable use include sterile aqueous solutions (where water soluble) or dispersions and sterile powders for the extemporaneous preparation of sterile injectable solutions or dispersion. Dispersions can be prepared in glycerol, liquid polyethylene glycols, and mixtures thereof and in oils. Under ordinary conditions of storage and use, these preparations may contain a preservative to prevent the growth of microorganisms. In all cases, the composition must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specifications for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The therapeutic compound can be orally administered, for example, with an inert diluent or an assimilable edible carrier. The therapeutic compound and other ingredients may also be enclosed in a hard- or soft-shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

Active compounds are administered at a therapeutically effective dosage sufficient to treat a condition associated with a condition in a patient. For example, the efficacy of a compound can be evaluated in an animal model system that may be predictive of efficacy in treating the disease in humans, such as the model systems shown in the examples and drawings.

The actual dosage amount of a compound of the present disclosure or composition comprising a compound of the present disclosure administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

An effective amount typically will vary from about 0.001 mg/kg to about 1000 mg/kg, from about 0.01 mg/kg to about 750 mg/kg, from about 100 mg/kg to about 500 mg/kg, from about 1.0 mg/kg to about 250 mg/kg, from about 10.0 mg/kg to about 150 mg/kg in one or more dose administrations daily, for one or several days (depending of course of the mode of administration and the factors discussed above). Other suitable dose ranges include 1 mg to 10000 mg per day, 100 mg to 10000 mg per day, 500 mg to 10000 mg per day, and 500 mg to 1000 mg per day. In some particular embodiments, the amount is less than 10,000 mg per day with a range of 750 mg to 9000 mg per day.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a pharmaceutical composition of the present disclosure may comprise, for example, at least about 0.1% of a compound of the present disclosure. In other embodiments, the compound of the present disclosure may comprise between about 2% to about 75% of the weight of the unit, or between about 25% to about 60%, for example, and any range derivable therein.

The agent(s) may be administered on a routine schedule. As used herein a routine schedule refers to a predetermined, designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the subject has eaten or will eat.

### VI. METHODS OF TREATMENT

Ovarian cancer begins when healthy cells in an ovary change and grow uncontrollably. Types of ovarian cancer include epithelial carcinomas, germ cell tumors, or stromal tumors. Epithelial carcinomas account for 85% to 90% of ovarian cancers. While historically considered to start on the surface of the ovary, new evidence suggests at least some ovarian cancers begin in special cells in the fallopian tube. Cancer cells that begin in the fallopian tube may go to the surface of the ovary early on. The term “ovarian cancer” is often used to describe epithelial cancers that begin in the ovary, in the fallopian tube, and from the lining of the abdominal cavity called the peritoneum.

At diagnosis, most women present with advanced disease, which accounts for the high mortality rate. Initial chemotherapy consists of either taxane or platinum chemotherapy or a combination of both. While approximately 75% of patients respond to front line therapy 70% of those eventually relapse within 1 to 3 years. As such, an ovarian cancer may have developed resistance to a platinum-based therapy. In some instances, an ovarian cancer may be a cisplatin-resistant ovarian cancer.

In some embodiments, the methods prolong progression-free survival as compared to control. In some embodiments, the methods reduce the hazard ratio for disease progression or death as compared to control. In some embodiments, the methods prolong overall survival as compared to control. In some embodiments, the methods achieve an overall response rate of at least 30%. In some embodiments, the methods achieve an improved chemotherapy-free interval as compared to control.

In certain embodiments, the compositions and methods of the present embodiments involve administration of SBP-101 and either eflornithine or doxorubicin. Therapeutic and prophylactic methods and compositions can be provided in a combined amount effective to achieve the desired effect. A tissue, organ, or cell can be exposed to a single composition or pharmacological formulation comprising both of the agents in a combination (i.e., SBP-101 and eflornithine, or SBP-101 and doxorubicin), or by contacting the tissue, organ, and/or cell with two or more distinct compositions or formulations, wherein one composition provides SBP-101 and another composition provides eflornithine or doxorubicin. Also, it is contemplated that such a combination therapy can be used in conjunction with surgical therapy.

SBP-101 may be administered before, during, after, or in various combinations relative to eflornithine or doxorubicin. The administrations may be in intervals ranging from concurrently to minutes to days to weeks. In embodiments where SBP-101 is provided to a patient separately from eflornithine or doxorubicin, one would generally ensure that a significant period of time did not expire between the time of each delivery, such that the two treatments would still be able to exert an advantageously combined effect on the patient. In such instances, it is contemplated that one may provide a patient with SBP-101 and either eflornithine or doxorubicin within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below, SBP-101 is “A” and doxorubicin or eflornithine is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A.

In some embodiments, the treatment methods may be supplemented with diagnostic methods to improve the efficacy and/or minimize the toxicity of the anti-cancer therapies comprising administration of the compositions provided herein. Such methods are described, for example, in U.S. Pat. Nos. 8,329,636 and 9,121,852, U.S. Patent Publications US2013/0217743 and US2015/0301060, and PCT Patent Publications WO2014/070767 and WO2015/195120, which are all incorporated herein by reference.

In some embodiments, compositions and formulations of the present disclosure may be administered to a subject with a genotype at position+316 (rs2302615) of at least one allele of the ODC1 gene promoter is G. In some embodiments, the genotype at position+316 of both alleles of the patient's ODC1 gene promoters may be GG. In some embodiments, the genotype at position+316 (rs2302615) of both alleles of the patient's ODC1 gene promoters may be GA. ODC1 A allele carriers at position+316 (rs2302615) differ in response to prolonged exposure with eflornithine and sulindac compared to GG genotype patients, with A allele carriers experiencing potential for elevated risk of developing ototoxicity, especially among the AA homozygotes. See U.S. Pat. No. 8,329,636, which is incorporated herein by reference.

In some embodiments, compositions and formulations of the present disclosure may be administered to a subject with a genotype at position+263 (rs2302616) of both alleles of the ODC1 gene is TT or TG. See PCT Patent Publication WO2015/195120, which is incorporated herein by reference.

### VII. COMBINATION TREATMENTS

In certain embodiments, the compositions and methods of the present embodiments involve administration of SBP-101 and either eflornithine or doxorubicin, and in further combination with a third or additional therapy.

### A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DFMO); retinoids, such as retinoic acid; capecitabine; carboplatin, procarbazine, plicomycin, gemcitabine, navelbine, farnesyl-protein transferase inhibitors, transplatinum, and pharmaceutically acceptable salts, acids, or derivatives of any of the above.

### B. Radiotherapy

Other factors that cause DNA damage and have been used extensively include what are commonly known as γ-rays, X-rays, and/or the directed delivery of radioisotopes to tumor cells. Other forms of DNA-damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### C. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T-cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune-stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds; cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, glucocorticoid-induced tumour necrosis factor receptor-related protein (GITR), HLA-DRB1, ICOS (also known as CD278), HLA-DQA1, HLA-E, indoleamine 2,3-dioxygenase 1 (IDO1), killer-cell immunoglobulin (KIR), lymphocyte activation gene-3 (LAG-3, also known as CD223), Mer tyrosine kinase (MerTK), NKG7, OX40 (also known as CD134), programmed death 1 (PD-1), programmed death-ligand 1 (PD-L1, also known as CD274), PDCD1LG2, PSMB10, STAT1, T-cell immunoreceptor with Ig and ITIM domains (TIGIT), T-cell immunoglobulin domain and mucin domain 3 (TIM-3), V-domain Ig suppressor of T-cell activation (VISTA, also known as C10orf54), and 4-1BB (CD137). In particular, the immune checkpoint inhibitors target the PD-1 axis and/or CTLA-4.

The immune checkpoint inhibitors may be drugs, such as small molecules, recombinant forms of ligand or receptors, or antibodies, such as human antibodies (e.g., International Patent Publication WO2015/016718; Pardoll, Nat Rev Cancer, 12 (4): 252-264, 2012; both incorporated herein by reference). Known inhibitors of the immune checkpoint proteins or analogs thereof may be used, in particular, chimeric, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen-binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T-cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T-cells, whereas CD28 transmits a stimulatory signal. Intracellular CTLA-4 is also found in regulatory T-cells and may be important to their function. T-cell activation through the T-cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art-recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95 (17): 10067-10071; Camacho et al. (2004) J Clin Oncology, 22 (145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 can also be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen-binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T-cells, natural killer cells, B cells, and plasmacytoid dendritic cells. LAG-3 acts as an “off” switch when bound to MHC class II on the surface of antigen-presenting cells. Inhibition of LAG-3 both activates effector T-cells and inhibitor regulatory T-cells. In some embodiments, the immune checkpoint inhibitor is an anti-LAG-3 antibody (e.g., a human antibody, a humanized antibody, or a chimeric antibody), an antigen-binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art-recognized anti-LAG-3 antibodies can be used. An exemplary anti-LAG-3 antibody is relatlimab (also known as BMS-986016) or antigen binding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T-cell activation (VISTA), also known as C10orf54. The complete protein sequence of human VISTA has the Genbank accession number NP_071436. VISTA is found on white blood cells and inhibits T-cell effector function. In some embodiments, the immune checkpoint inhibitor is an anti-VISTA3 antibody (e.g., a human antibody, a humanized antibody, or a chimeric antibody), an antigen-binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art-recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is indoleamine 2,3-dioxygenase (IDO). The complete protein sequence of human IDO has Genbank accession number NP_002155. In some embodiments, the immune checkpoint inhibitor is a small molecule IDO inhibitor. Exemplary small molecules include BMS-986205, epacadostat (INCB24360), and navoximod (GDC-0919).

Another immune checkpoint protein that can be targeted in the methods provided herein is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen-binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art-recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen-binding fragment thereof, an immunoadhesin, a fusion protein, or oligopeptide. Anti-human-ICOS antibodies (or VH and/or VL domains derived therefrom) suitable for use in the present methods can be generated using methods well known in the art. Alternatively, art-recognized anti-ICOS antibodies can be used. Exemplary anti-ICOS antibodies include JTX-2011 (see, e.g., WO 2016/154177, WO 2018/187191) and GSK3359609 (see, e.g., WO 2016/059602).

Another immune checkpoint protein that can be targeted in the methods provided herein is T-cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen-binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art-recognized anti-TIGIT antibodies can be used. An exemplary anti-TIGIT antibody is MK-7684 (see, e.g., WO 2017/030823, WO 2016/028656).

Another immune checkpoint protein that can be targeted in the methods provided herein is OX40, also known as CD134. The complete protein sequence of human OX40 has Genbank accession number NP_003318. In some embodiments, the immune checkpoint inhibitor is an anti-OX40 antibody (e.g., a human antibody, a humanized antibody, or a chimeric antibody), an antigen-binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art-recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another immune checkpoint protein that can be targeted in the methods provided herein is glucocorticoid-induced tumor necrosis factor receptor-related protein (GITR), also known as TNFRSF18 and AITR. The complete protein sequence of human GITR has Genbank accession number NP_004186. In some embodiments, the immune checkpoint inhibitor is an anti-GITR antibody (e.g., a human antibody, a humanized antibody, or a chimeric antibody), an antigen-binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art-recognized anti-GITR antibodies can be used. An exemplary anti-GITR antibody is TRX518 (see, e.g., WO 2006/105021).

Another immune checkpoint protein that can be targeted in the methods provided herein is T-cell immunoglobulin and mucin-domain containing-3 (TIM3), also known as HAVCR2. The complete protein sequence of human TIM3 has Genbank accession number NP_116171. In some embodiments, the immune checkpoint inhibitor is an anti-TIM3 antibody (e.g., a human antibody, a humanized antibody, or a chimeric antibody), an antigen-binding fragment thereof, an immunoadhesin, a fusion protein, or oligopeptide. Anti-human-TIM3 antibodies (or VH and/or VL domains derived therefrom) suitable for use in the present methods can be generated using methods well known in the art. Alternatively, art-recognized anti-TIM3 antibodies can be used. Exemplary anti-TIM3 antibodies include LY3321367 (see, e.g., WO 2018/039020), MBG453 (see, e.g., WO 2015/117002) and TSR-022 (see, e.g., WO 2018/085469).

Another immune checkpoint protein that can be targeted in the methods provided herein is 4-1BB, also known as CD137, TNFRSF9, and ILA. The complete protein sequence of human 4-1BB has Genbank accession number NP_001552. In some embodiments, the immune checkpoint inhibitor is an anti-4-1BB antibody (e.g., a human antibody, a humanized antibody, or a chimeric antibody), an antigen-binding fragment thereof, an immunoadhesin, a fusion protein, or oligopeptide. Anti-human-4-1BB antibodies (or VH and/or VL domains derived therefrom) suitable for use in the present methods can be generated using methods well known in the art. Alternatively, art-recognized anti-4-1BB antibodies can be used. An exemplary anti-4-1BB antibody is PF-05082566 (utomilumab; see, e.g., WO 2012/032433).

In some embodiment, the immune therapy could be adoptive immunotherapy, which involves the transfer of autologous antigen-specific T-cells generated ex vivo. The T-cells used for adoptive immunotherapy can be generated either by expansion of antigen-specific T-cells or redirection of T-cells through genetic engineering. Isolation and transfer of tumor-specific T cells has been shown to be successful in treating melanoma. Novel specificities in T-cells have been successfully generated through the genetic transfer of transgenic T-cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T-cells to be redirected against antigens expressed at the surface of tumor cells from various malignancies including lymphomas and solid tumors.

In one embodiment, the present application provides for a combination therapy for the treatment of cancer wherein the combination therapy comprises adoptive T-cell therapy and a checkpoint inhibitor. In one aspect, the adoptive T-cell therapy comprises autologous and/or allogeneic T-cells. In another aspect, the autologous and/or allogeneic T-cells are targeted against tumor antigens. The engineered hSDH enzyme may be administered to the patient prior to and/or simultaneously with the administration of the adoptive T-cell therapy. In another aspect, the autologous and/or allogeneic T-cells may be engineered to express the engineered hSDH enzyme.

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically controlled surgery (Mohs' surgery).

Upon excision of part or all of the cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VIII. KITS

Provided are kits, such as therapeutic kits. For example, a kit may comprise one or more therapeutic composition as described herein and optionally instructions for their use. Kits may also comprise one or more devices for accomplishing administration of such compositions. For example, a subject kit may comprise a therapeutic composition and catheter for accomplishing direct intravenous injection of the composition into a target tissue. A kit may comprise pre-filled ampoules of an engineered hSDH enzyme, optionally formulated as a therapeutic composition, or lyophilized, for use with a delivery device.

Kits may comprise a container with a label. Suitable containers include, for example, bottles, vials, and test tubes. The containers may be formed from a variety of materials, such as glass or plastic. The container may hold a composition that includes an engineered hSDH enzyme that is effective for therapeutic or non-therapeutic applications, such as described above. The label on the container may indicate that the composition is used for a specific therapy or non-therapeutic application and may also indicate directions for either in vivo or in vitro use, such as those described above. Kits will typically comprise the container described above and one or more other containers comprising materials desirable from a commercial and user standpoint, including buffers, diluents, filters, needles, syringes, and package inserts with instructions for use.

### IX. EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Example 1—Combination with SBP-101 and Gemcitabine

Cell lines, culture conditions, and reagents. Ovarian adenocarcinoma cell lines A2780 and ACRP were maintained in RPMI 1640 containing 10% fetal bovine serum. Ovarian adenocarcinoma lines CaOV-3 and OV90 were maintained in DMEM supplemented with 10% fetal bovine serum. ID8 mouse ovarian surface epithelial cells (MOSE) overexpressing VEGF and β-Defensin (VDID8+) were maintained in RPMI 1640 supplemented with 10% fetal bovine serum. The polyamine analogue ivospemin (SBP-101) was obtained from Panbela Therapeutics, Inc (Waconia, MN). All chemotherapeutics were purchased commercially from Sigma-Aldrich (St. Louis, MO).

Cell viability assays. Cells were seeded in triplicate wells per condition with a total cell count of 2×10³ cells per well of a 96-well plate and allowed to adhere overnight. Cells then received 100 μL fresh medium with increasing concentrations of ivospemin. Following 96 hours of ivospemin monotherapy, 20 μL of CellTiter-Blue reagent (Promega, Madison, WI) was added, and following a 3-hour incubation, the fluorescence was measured in black, clear-bottom plates at 560_{Ex}/590_{Em} on a SpectraMax M5 (Molecular Devices, Sunnyvale CA). Wells containing medium alone were used as background controls. The combination chemotherapy/ivospemin experiment conditions were identical except following 72 hours of ivospemin exposure, cells received fresh medium with ivospemin and the appropriate chemotherapeutic. After an additional 24 hours (96 total hours of ivospemin, 24 hours of chemotherapy), cell viability was determined using the CellTiter-Blue cell viability assay. Results are presented as percentages relative to untreated cells. Each bar is an average of at least 3 independent biological experiments (each experiment is noted by an individual point).

Syngeneic mouse model. Female C57BL/6J wild-type mice (7-8 weeks old, The Jackson Laboratory) were housed at the Johns Hopkins Sidney Kimmel Comprehensive Cancer Center Animal Resources Core and cared for in accordance with the policies set forth by the Johns Hopkins University Animal Care and Use Committee. VEGF-β-Defensin ID8 (VDID8+) syngeneic mouse ovarian surface epithelial cells (first developed by Dr. Katherine Roby) were injected intraperitoneally into C57BL/6 mice (350,000 cells/mouse) (Roby et al., 2000). Treatment of mice began three days following VDID8⁺ injection. Mice in the combination study were treated with the following dosing schedule: 24 mg/kg ivospemin 2qw×3, alternating weeks; 30 mg/kg gemcitabine qw×4; 1 mg/kg topotecan 3qw×4. All drugs were administered intraperitoneally. Following production of palpable ascites fluid or a 15% weight gain, animals were drained of their ascites fluid, which was measured as a marker for tumor burden. Decisions for subsequent drains was determined by the same criteria. No animals were drained more than once a week, and animals who survived to a fourth drain were subsequently euthanized.

Statistical analysis. All statistical testing was completed using GraphPad Prism software (v9.5.1, La Jolla, CA). Dose responses of ovarian cells to ivospemin were plotted by a non-linear regression to determine IC₅₀ values. Cell viability experiments were analyzed by one-way ANOVA and then individual comparisons were analyzed by two-sided Welch's t-tests unless otherwise specified. Multiple testing correction was performed based on the Benjamini-Hochberg method. All data passed the Shapiro-Wilk test for normality. All mouse survival curves were analyzed using the log-rank (Mantel-Cox) test. Mouse ascites data were analyzed by one-way ANOVA and then individual comparisons were analyzed by two-sided Welch's t-tests unless otherwise specified. Multiple testing correction was performed based on the Benjamini-Hochberg method. All data passed the Shapiro-Wilk test for normality. A p-value of <0.05 was considered statistically significant. P-value indications are as follows: *<0.05; **<0.01; ***<0.001; ****<0.0001.

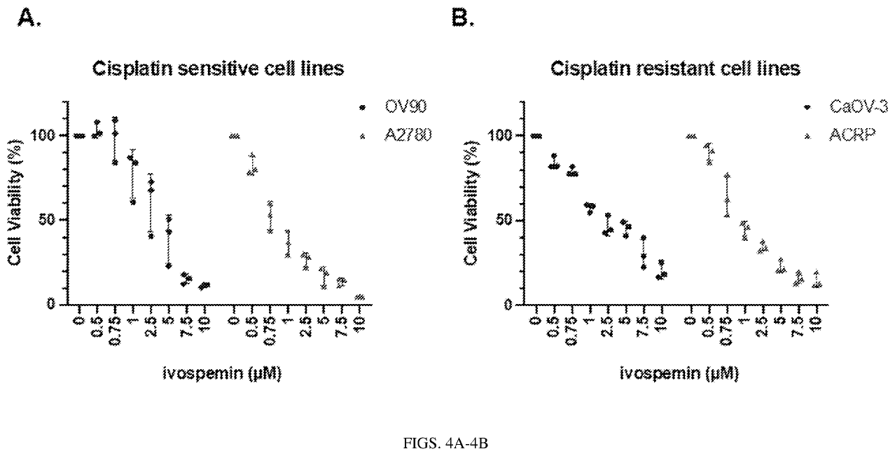

Ivospemin co-treatment increases chemotherapy toxicity in ovarian adenocarcinoma cell lines regardless of cisplatin sensitivity. The cisplatin-sensitive ovarian adenocarcinoma lines, A2780 and OV90 (FIG. 4A), and the cisplatin-resistant ovarian adenocarcinoma lines, ACRP and CaOV-3 (FIG. 4B), were treated for 96 hours with concentrations of ivospemin ranging from 500 nM to 10 μM. All four lines, regardless of cisplatin sensitivity, responded to ivospemin treatment with IC₅₀ values ranging between 0.95 μM and 3.24 μM (Table 1). These four ovarian adenocarcinoma lines were then treated with various chemotherapeutic agents including gemcitabine (50 nM), topotecan (50 nM), paclitaxel (2 nM) and docetaxel (2 nM). These agents represent standard of care chemotherapeutic options used with some regularity in patients with platinum-resistant ovarian cancer. Cells were treated with three increasing concentrations of each chemotherapy alone to determine an appropriate dose prior to combination studies.

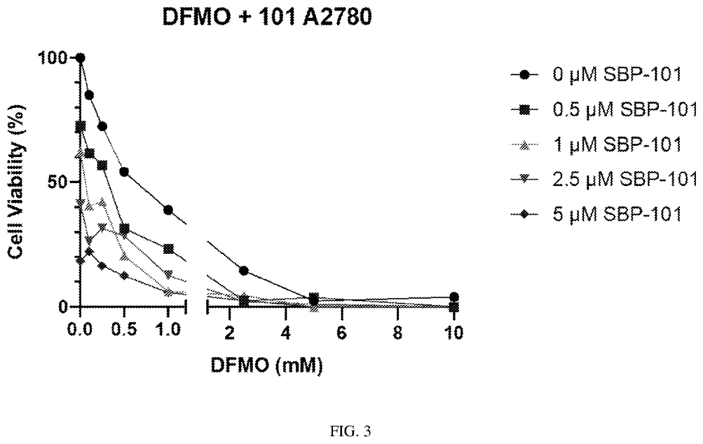

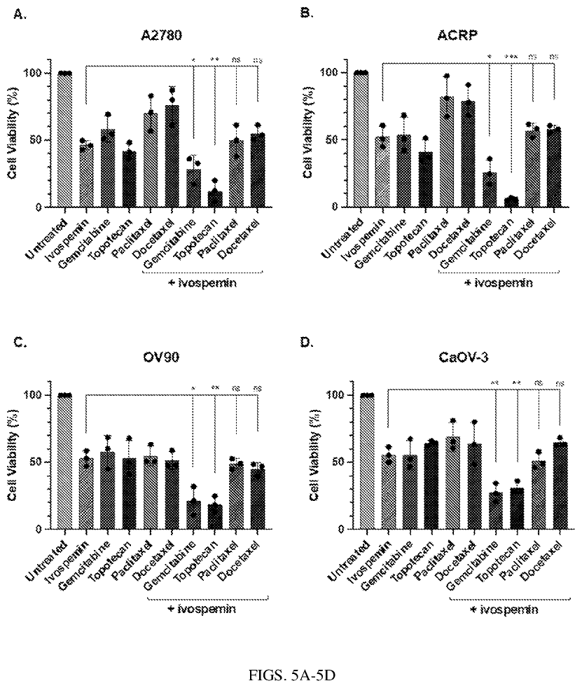

Each ovarian adenocarcinoma line was treated with 2 μM ivospemin in addition to each chemotherapeutic agent (FIGS. 5A-5D). The toxicity of each chemotherapeutic agent was increased by ivospemin co-treatment in A2780 cells, however, only in combinations with gemcitabine or topotecan was this toxicity increase statistically significant over that of either agent alone (p-values of 0.045 and 0.0023, respectively, compared to ivospemin treatment) (FIG. 3A). Similarly, ivospemin co-treatment increased the toxicity of each chemotherapeutic agent in ACRP cells with the reciprocal treatment of gemcitabine and topotecan resulting in greater toxicity than ivospemin alone (p-values of 0.02 and 0.0006, respectively) (FIG. 3B). Ivospemin did not increase the toxicity of paclitaxel or docetaxel in OV90 cells, it but did increase toxicity of both gemcitabine and topotecan (p-values of 0.01 and 0.002, respectively) (FIG. 3C). Similarly, ivospemin treatment only significantly increased the toxicity of gemcitabine and topotecan in CaOV-3 cells (p-values of 0.005 and 0.004, respectively) (FIG. 3D). The addition of ivospemin increases the toxicity of each evaluated chemotherapeutic agent. In each cell line, ivospemin increases both topotecan and gemcitabine's toxicity in a statistically significant manner (Table 2). While cell viability was decreased in all cell lines following the addition of ivospemin to either paclitaxel or docetaxel, only a subset of cell lines decreased viability (Table 2). Overall, gemcitabine and topotecan comparably increased ivospemin toxicity to all four ovarian adenocarcinoma lines tested, while paclitaxel and docetaxel treatment consistently added little to no benefit to ivospemin monotherapy in any of the tested cell lines. However, ivospemin addition improved the response to each of the four tested chemotherapeutic agents.

TABLE 1
IC₅₀ values following ivospemin treatment in ovarian
adenocarcinoma cell lines

| Cell Line | Cisplatin Sensitivity | IC₅₀ |
|---|---|---|
| OV90 | Sensitive | 3.24 μM |
| A2780 | Sensitive | 0.95 μM |
| CaOV-3 | Resistant | 2.56 μM |
| ACRP | Resistant | 1.41 μM |

TABLE 2
Individual comparisons of ivospemin combination treatment vs.
chemotherapeutic monotherapy

| Comparison | P-value | Significance |
|---|---|---|
| Gemcitabine (A2780) | 0.021 | * |
| Gemcitabine (ACRP) | 0.041 | * |
| Gemcitabine (CaOV-3) | 0.018 | * |
| Gemcitabine (OV90) | 0.016 | * |
| Topotecan (A2780) | 0.007 | ** |
| Topotecan (ACRP) | 0.003 | ** |
| Topotecan (CaOV-3) | 0.0003 | *** |
| Topotecan (OV90) | 0.016 | * |
| Paclitaxel (A2780) | 0.114 | ns |
| Paclitaxel (ACRP) | 0.054 | ns |
| Paclitaxel (CaOV-3) | 0.025 | * |
| Paclitaxel (OV90) | 0.281 | ns |
| Docetaxel (A2780) | 0.034 | * |
| Docetaxel (ACRP) | 0.035 | * |
| Docetaxel (CaOV-3) | 0.338 | ns |
| Docetaxel (OV90) | 0.243 | ns |

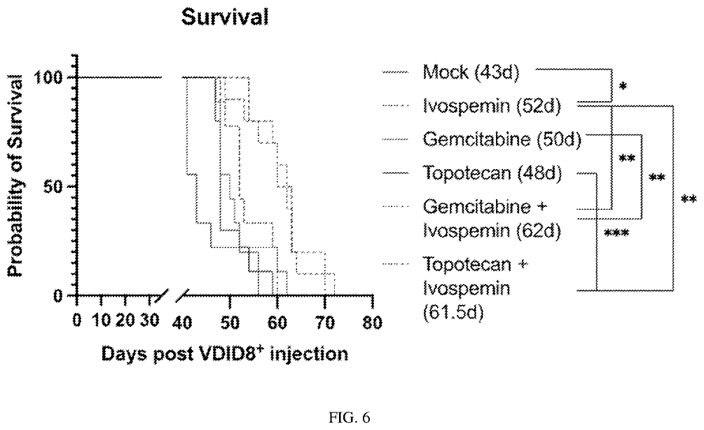

Ivospemin treatment in combination with chemotherapy increases survival in a murine ovarian adenocarcinoma model compared to chemotherapy alone. To further evaluate the potential benefit of ivospemin addition to gemcitabine and topotecan in vivo, female C57Bl/6J mice were injected with 350,000 VDID8⁺ syngeneic ovarian epithelial cancer cells and subsequently treated with the appropriate drugs. Ivospemin alone produced a 20% increase in median survival when compared to control animals (FIG. 6). Neither gemcitabine nor topotecan produced a statistically significant survival benefit as a monotherapy (Table 3). The addition of ivospemin to topotecan increased survival by 28% compared to topotecan monotherapy, and the addition of ivospemin to gemcitabine increased survival by 24% compared to gemcitabine alone (FIG. 6). Treatment with either double combination resulted in an approximately 45% increase in median survival compared to untreated animals.

TABLE 3
Individual comparisons of median survival. Gemcitabine
(gem), ivospemin (ivo) and topotecan (topo)

| Comparison | P-value | Significance |
|---|---|---|
| Mock vs Ivo | 0.026 | * |
| Mock vs Gem | 0.053 | ns |
| Mock vs Topo | 0.170 | ns |
| Ivo vs Gem | 0.559 | ns |
| Ivo vs Topo | 0.037 | * |
| Ivo vs Gem + Ivo | 0.004 | ** |
| Ivo vs Topo + Ivo | 0.003 | ** |
| Gem vs Gem + Ivo | 0.004 | ** |
| Topo vs Topo + Ivo | 0.0002 | *** |

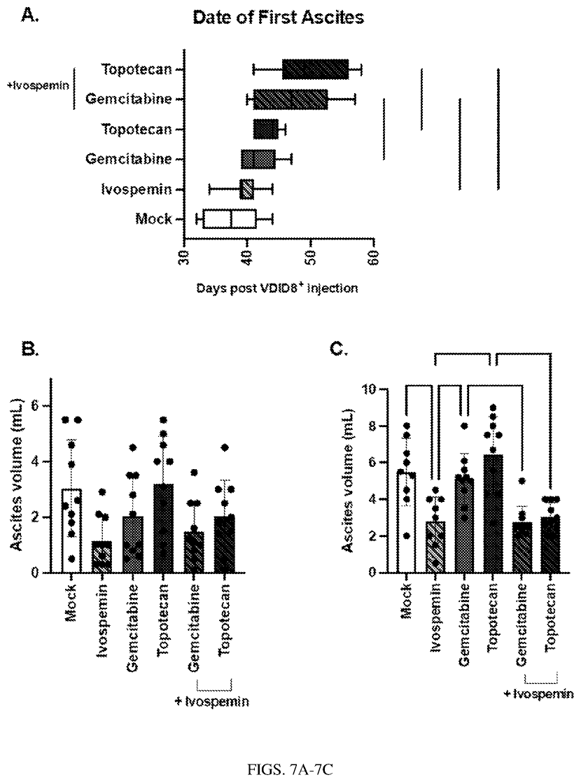

Ivospemin increases chemotherapeutic efficacy by delaying disease onset and decreasing overall tumor burden in a murine ovarian adenocarcinoma model. There is an average of 37.5 days between VDID8+ cell injection and measurable ascites formation in untreated animals as determined by either 15% weight gain or visible abdominal swelling (FIG. 7A). Ivospemin alone does not significantly affect the time to ascites formation, with an average time of 39.5 days. Gemcitabine and topotecan each modestly delayed ascites formation with averages times of 41.7 and 43.4 days, respectively. The addition of ivospemin to gemcitabine extended the time to ascites formation to 46.9 days, an increase of 13%, while ivospemin and topotecan combination mice exhibited ascites 50 days post VDID8⁺ injection, a 15% increase (FIG. 7A).

Untreated mice produced an average of 3 mL of ascites fluid at their first drain (FIG. 7B). Ivospemin monotherapy appeared to decrease overall ascites production (a measure of tumor burden) with an average ascites volume at first drain of 1.15 mL, though this decrease did not quite reach the level of statistical significance (p-value=0.0541). Similarly, gemcitabine and topotecan monotherapies had no impact on ascites formation, with average first drain volumes of 2.03 and 3.19 mL, respectively (FIG. 7B). The addition of ivospemin to either monotherapy trended toward decreasing average ascites fluid but did not reach statistical significance at first drain (FIG. 7B). However, ivospemin maintained its influence on ascites volume through subsequent drains, while the monotherapy volumes increased. Ivospemin-treated mice produced 50% less ascites than untreated mice at their second drain (p-value=0.0027) (FIG. 7C), and its addition to the chemotherapies also reduced ascites volume: adding ivospemin to gemcitabine or topotecan reduced ascites formation by 46% (p-value=0.006) and 53% (p-value=<0.0001), respectively (FIG. 7C).

### Example 2—Combination with SBP-101 and Doxorubicin

SBP-101 treatment decreases overall polyamine content through depression of the activity of the polyamine biosynthetic enzyme ornithine decarboxylase (ODC) in a variety of cancer cell lines including ovarian cancer. Treatment of the VDID8⁺ murine ovarian cancer model with SBP-101 resulted in a marked increase in median survival.

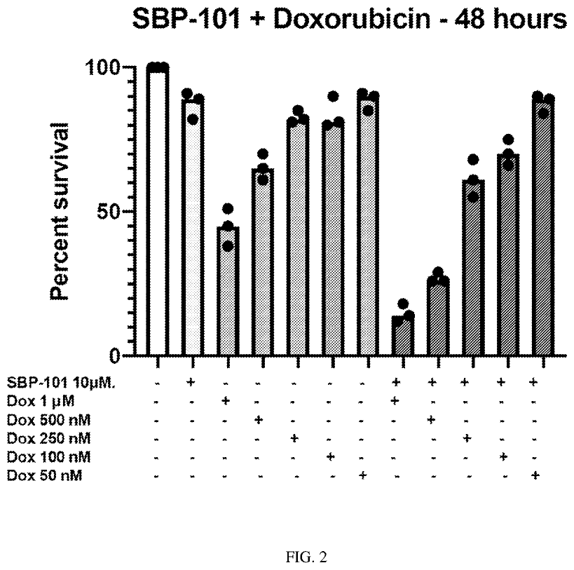

The potential synergy between SBP-101 and five chemotherapeutics (gemcitabine, topotecan, doxorubicin, paclitaxel, and docetaxel) has been further evaluated in vitro. These chemotherapies are all used, with some regularity, to treat platinum-resistant ovarian cancer patients. Treatment with gemcitabine, topotecan, and doxorubicin increased the in vitro toxicity of SBP-101 in both cisplatin-sensitive and cisplatin-resistant ovarian cancer cell lines (FIG. 2, A2780 ovarian cells treated with doxorubicin). Paclitaxel and docetaxel did not have any added benefit in vitro to SBP-101 alone.

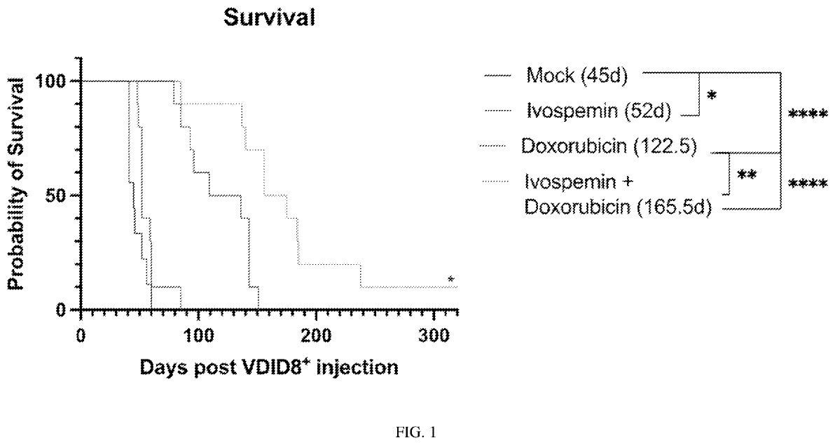

Using the immunosuppressive VDID8⁺ murine ovarian cancer model, the efficacy of SBP-101 in combination with either gemcitabine, topotecan, or doxorubicin was further evaluated. Ascites production was recorded as a marker of tumor burden and ascites fluid was collected for evaluation of polyamine content in the tumor microenvironment. Gemcitabine and topotecan alone had little effect on overall survival of the mice, whereas either SBP-101 or doxorubicin treatment alone significantly increased median mouse survival time. Addition of SBP-101 improved the survival of mice treated with any of the three chemotherapeutics. The SBP-101 and doxorubicin combination mice had the greatest median survival time (FIGS. 1 & 6, doxorubicin dosage of 1 mg/kg MWF for 4 weeks=12 total doses=approximately 250 μg total). The addition of SBP-101 to 1 mg/kg (t.i.w for 4 weeks) doxorubicin treatment improved median survival by 50% with a p-value of 0.001. The combination is being further evaluated in mechanistic studies as well as subsequent murine studies.

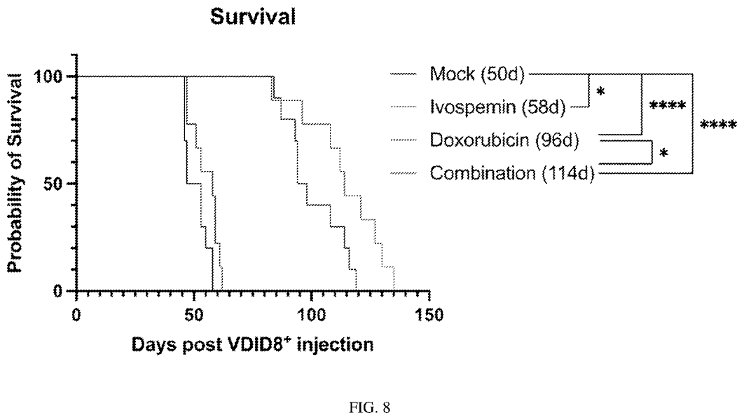

Using the VDID8⁺ murine model, the efficacy of SBP-101 in combination with low dose doxorubicin was evaluated with a doxorubicin dosage of 0.5 mg/kg twice per week for 4 weeks (=8 doses=approximately 80 μg total). This results in a 68% reduction in total doxorubicin exposure and represents a subclinical dosing level (FIG. 8). The addition of SBP-101 to 0.5 mg/kg doxorubicin treatment improved medial survival by 20% with a p-value of 0.03, and the combination treatment resulted in a 128% increase in medial survival over untreated animals (p-value=<0.0001).

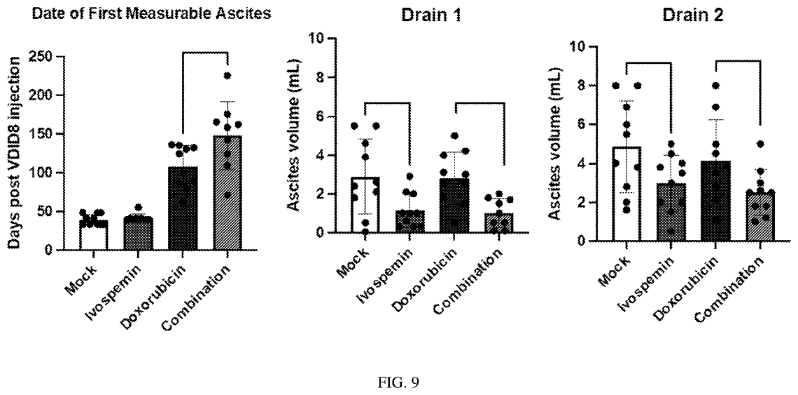

Mice co-treated with SBP-101 (24 mg/kg 2qw alt week) and subclinical doxorubicin (0.5 mg/kg 2qw×4) benefited from an approximate 16.8 day delay in ascites onset (approximately 20%) compared to doxorubicin single agent animals with a p-value of 0.04. The addition of SBP-101 to low dose doxorubicin also decreased overall tumor burden as measured by ascites (FIG. 9). Combination treated mice exhibited a 50% reduction in ascites volume at first drain (p-value of 0.019) and a 62% reduction in ascites volume at second drain (p-value of 0.007).

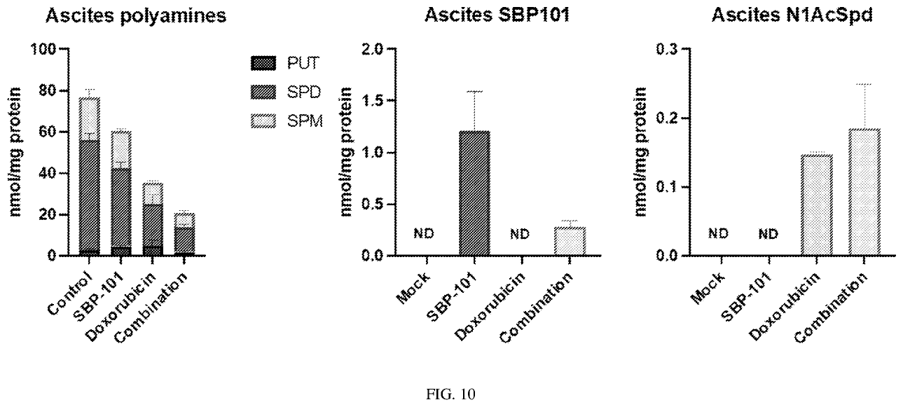

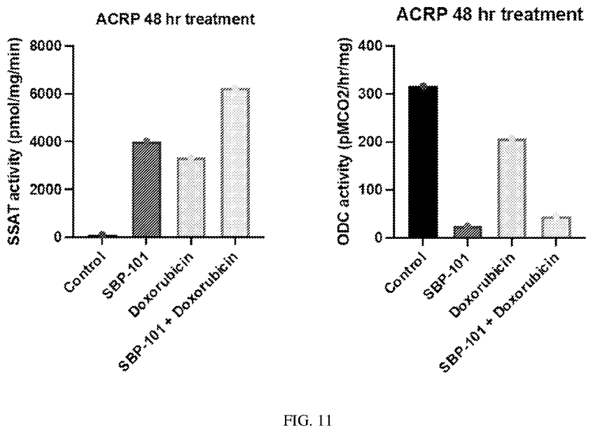

Ascites fluid was collected from the second drain of animals treated with ivospemin and low-dose doxorubicin. Red blood cells were lysed and the remaining cells (both tumor and microenvironment cells such as immune and stromal) were pelleted and acid extracted for quantification of polyamine content by high performance liquid chromatography. Combination treated animals had the lowest ascitic polyamine content, exhibiting a 73% reduction in polyamine content compared to mock (p-value=0.0016) and a 40% reduction compared to doxorubicin alone (p-value=0.02) (FIG. 10). As expected, SBP-101 could be detected by HPLC in SBP-101 treated animals only. N1-acetylated spermidine could be detected in doxorubicin and combination ascites fluid indicating a possible upregulation of SSAT.

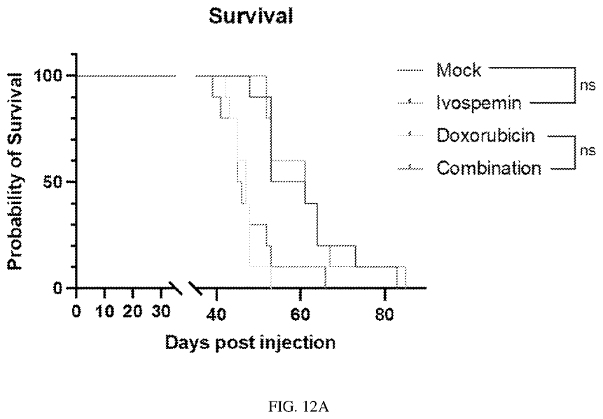

The human ovarian adenocarcinoma line was treated with SBP-101 (5 μM) and doxorubicin (500 nM) for 48 hours. The activity of polyamine catabolic enzyme SSAT and polyamine biosynthetic enzyme ODC were evaluated. Either SBP-101 or doxorubicin increase SSAT activity with combination treatment having the highest catabolic activity (FIG. 11). SBP-101 decreases ODC activity alone or in combination with doxorubicin, however the combination does not have any additional ODC inhibitory effect. This suggests that the combinatorial benefit observed may be partially explained by additive SSAT induction.

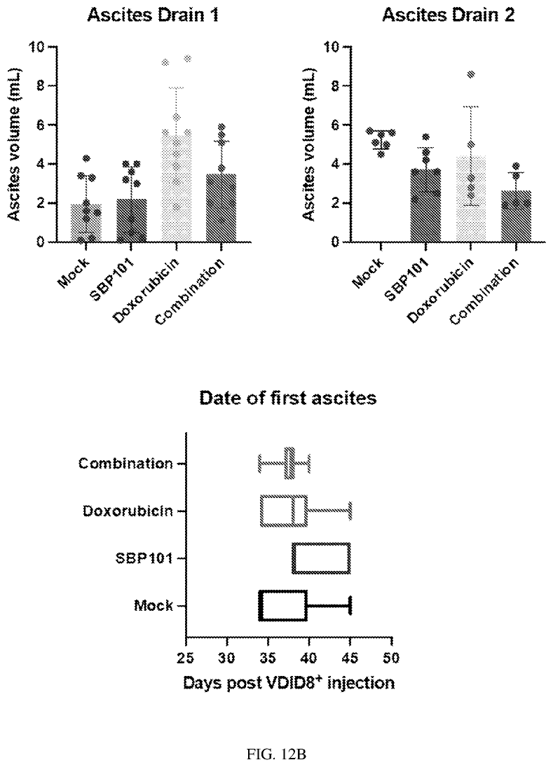

Efficacy of the combination treatment is dependent on an intact immune system. Immunocompromised NSG mice were injected with 250,000 VDID8⁺ cells per mouse and subsequently treated with 24 mg/kg 2qw alt weeks ivospemin (SBP-101) and a subclinical dose of doxorubicin (0.5 mg/kg 2qw×4). Even at a subclinical dosing level, doxorubicin alone produced some toxicities among treated animals, most notably weight loss and anemia, however none of the three treatment arms resulted in a survival benefit (FIG. 12A) nor did treatment decrease tumor burden or delay ascites formation as previously seen (FIG. 12B).

### Example 3—Combination with SBP-101 and DFMO

A sustained elevation of polyamine levels plays a role in the immunosuppressive environment of some cold tumors, and the pharmacologic and genetic modulation of polyamine metabolism have demonstrated success in reducing immunosuppressive phenotypes. Of note, DFMO (difluoromethylornithine/eflornithine) treatment in combination with an epigenetic therapy in the VDID8⁺ murine ovarian cancer model has shown increased survival and a reprogramming of the tumor microenvironment to a more immune-friendly state. SBP-101 and DFMO treatment in ovarian cancer have a cooperative effect in vitro (FIG. 3). Future studies will determine cooperativity between these two drugs in vivo and any influences on the immune microenvironment.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 6,160,022
U.S. Pat. No. 8,329,636
U.S. Pat. No. 9,121,852
U.S. Patent Publication 2013/0217743
U.S. Patent Publication 2015/0301060
PCT Patent Publication WO2014/070767
PCT Patent Publication WO2015/195120
PCT Patent Publication WO2019/152323
Bello-Fernandez, C., G. Packham, and J. L. Cleveland, The ornithine decarboxylase gene is a transcriptional target of c-Myc. Proc Natl Acad Sci USA, 1993. 90 (16): p. 7804-8.
Bergeron, R. J., et al., Synthesis and evaluation of hydroxylated polyamine analogues as antiproliferatives. J Med Chem, 2000. 43 (2): p. 224-35.
Bergeron, R. J., et al., Synthetic polyamine analogues as antineoplastics. J Med Chem, 1988. 31 (6): p. 1183-90.
Casero, R. A. and A. E. Pegg, Polyamine catabolism and disease. Biochem J, 2009. 421 (3): p. 323-38.
Casero, R. A. and L. J. Marton, Targeting polyamine metabolism and function in cancer and other hyperproliferative diseases. Nat Rev Drug Discov, 2007. 6 (5): p. 373-90.
Casero, R. A. and P. M. Woster, Terminally alkylated polyamine analogues as chemotherapeutic agents. J Med Chem, 2001. 44 (1): p. 1-26.
Casero, R. A., T. Murray Stewart, and A. E. Pegg, Polyamine metabolism and cancer: treatments, challenges and opportunities. Nat Rev Cancer, 2018. 18 (11): p. 681-695. Creaven, P. J., et al., Unusual central nervous system toxicity in a phase I study of N1N11 diethylnorspermine in patients with advanced malignancy. Invest New Drugs, 1997. 15 (3): p. 227-34.
Cronin, K. A., et al., Annual report to the nation on the status of cancer, part 1: National cancer statistics. Cancer, 2022. 128 (24): p. 4251-4284.
da Costa, A. A. B. A. and G. Baiocchi, Genomic profiling of platinum-resistant ovarian cancer: The road into druggable targets. Semin Cancer Biol, 2021. 77: p. 29-41.
Fogel-Petrovic, M., et al., Differential post-transcriptional control of ornithine decarboxylase and spermidine-spermine N1-acetyltransferase by polyamines. FEBS Lett, 1996. 391 (1-2): p. 89-94.
Fogel-Petrovic, M., et al., Effects of polyamines, polyamine analogs, and inhibitors of protein synthesis on spermidine-spermine N1-acetyltransferase gene expression. Biochemistry, 1996. 35 (45): p. 14436-44.
Gerner, E. W. and F. L. Meyskens, Polyamines and cancer: old molecules, new understanding. Nat Rev Cancer, 2004. 4 (10): p. 781-92.
Ghoneum, A., et al., Exploring the clinical value of tumor microenvironment in platinum-resistant ovarian cancer. Semin Cancer Biol, 2021. 77: p. 83-98.
Hahm, H. A., et al., Phase I study of N (1),N (11)-diethylnorspermine in patients with non-small cell lung cancer. Clin Cancer Res, 2002. 8 (3): p. 684-90.
Holbert, C. E., et al., Expanded Potential of the Polyamine Analogue SBP-101 (Diethyl Dihydroxyhomospermine) as a Modulator of Polyamine Metabolism and Cancer Therapeutic. Int J Mol Sci, 2022. 23 (12).
Muñoz-Galván, S. and A. Carnero, Targeting Cancer Stem Cells to Overcome Therapy Resistance in Ovarian Cancer. Cells, 2020. 9 (6).
Murray Stewart, T., et al., A phase I dose-escalation study of the polyamine analog PG-11047 in patients with advanced solid tumors. Cancer Chemother Pharmacol, 2020a. 85 (6): p. 1089-1096.
Murray Stewart, T., et al., A Phase Ib multicenter, dose-escalation study of the polyamine analogue PG-11047 in combination with gemcitabine, docetaxel, bevacizumab, erlotinib, cisplatin, 5-fluorouracil, or sunitinib in patients with advanced solid tumors or lymphoma. 2020b: Cancer Chemother. Pharmacol.
Pegg, A. E. and R. H. Hu, Effect of polyamine analogues and inhibition of polyamine oxidase on spermidine/spermine N1-acetyltransferase activity and cell proliferation. Cancer Lett, 1995. 95 (1-2): p. 247-52.
Pegg, A. E., Mammalian polyamine metabolism and function. IUBMB Life, 2009. 61 (9): p. 880-94.
Roby, K. F., et al., Development of a syngeneic mouse model for events related to ovarian cancer. Carcinogenesis, 2000. 21 (4): p. 585-91.
Shah, A., M. Cullen, and C. Baker, Abstract 3128: Efficacy of diethyldihydroxyhomospermine against human pancreatic adenocarcinoma using orthotopic implantation of human pancreatic L3.6pl cells into the pancreas of nude mice. 2014: Cancer Research.
Singhal, N., et al., SBP-101, a polyamine metabolic inhibitor, administered in combination with gemcitabine and nab-paclitaxel, shows signals of efficacy as first-line treatment for subjects with metastatic pancreatic ductal adenocarcinoma. Journal of Clinical Oncology, 2021. 39 (15 suppl).
Sung, H., et al., Global Cancer Statistics 2020: GLOBOCAN Estimates of Incidence and Mortality Worldwide for 36 Cancers in 185 Countries. CA Cancer J Clin, 2021. 71 (3): p. 209-249.
Therapeutics, P. Phase 1A/1B Dose Escalation and Expansion Study of SBP-101 in Combination With Nab-Paclitaxel and Gemcitabine in Subjects With Previously Untreated Metastatic Pancreatic Ductal Adenocarcinoma. Updated May 25, 2022 [cited 2023 May 12]; ClinicalTrials.gov identifier: NCT03412799: [
Therapeutics, P., A Randomized, Double-Blind, Placebo-Controlled Study of Nab-Paclitaxel and Gemcitabine With or Without SBP-101 in Subjects Previously Untreated for Metastatic Pancreatic Ductal Adenocarcinoma. Updated Apr. 4, 2023, ClinicalTrials.gov identifier: NCT05254171.
Thomas, T. and T. J. Thomas, Polyamine metabolism and cancer. J Cell Mol Med, 2003. 7 (2): p. 113-26.
Wilding, G., et al., Phase I trial of the polyamine analog N1,N14-diethylhomospermine (DEHSPM) in patients with advanced solid tumors. Invest New Drugs, 2004. 22 (2): p. 131-8.

1. A method of treating a patient having an ovarian cancer, the method comprising administering to the patient a combined effective amount of SBP-101 and doxorubicin.

2. The method of claim 1, wherein the ovarian cancer in the subject was previously treated with the anti-cancer agent or at least one other anti-cancer agent.

3. The method of claim 1 or 2, wherein the ovarian cancer has developed resistance to a platinum-based therapy.

4. The method of claim 1, wherein the ovarian cancer is a platinum-resistant ovarian cancer.

5. The method of any one of claims 1-4, further comprising administering at least one additional therapeutic to the subject.

6. The method of claim 5, wherein the at least one additional therapeutic is selected from the group consisting of surgical therapy, chemotherapy, radiation therapy, cryotherapy, hormonal therapy, toxin therapy, immunotherapy, or cytokine therapy.

7. The method of any one of claims 1-6, wherein the SBP-101 and doxorubicin are administered consecutively.

8. The method of claim 7, wherein the SBP-101 is administered prior to the doxorubicin.

9. The method of claim 7, wherein the SBP-101 is administered after the doxorubicin.

10. The method of any one of claims 1-6, wherein the SBP-101 and doxorubicin are administered simultaneously.

11. The method of claim 1 or 10, wherein the SBP-101 and doxorubicin are co-formulated.

12. The method of any one of claims 1-11, wherein the patient is human.

13. A pharmaceutical composition comprising SBP-101, doxorubicin, and a pharmaceutically acceptable carrier.

14. The pharmaceutical composition of claim 13, for use in the treatment of ovarian cancer.

15. A pharmaceutical composition comprising SBP-101, for use in the treatment of ovarian cancer in combination with a second pharmaceutical therapy comprising doxorubicin.

16. A pharmaceutical composition comprising doxorubicin, for use in the treatment of ovarian cancer in combination with a second pharmaceutical therapy comprising SBP-101.

17. The pharmaceutical composition for use of claim 15 or 16, wherein the ovarian cancer is a platinum-resistant ovarian cancer.

18. Use of SBP-101 in the manufacture of a medicament for the treatment of ovarian cancer in combination with a second medicament comprising doxorubicin.

19. Use of doxorubicin in the manufacture of a medicament for the treatment of ovarian cancer in combination with a second medicament comprising SBP-101.

20. Use of SBP-101 and doxorubicin in the manufacture of a medicament for the treatment of ovarian cancer.

21. The use of any one of claims 18-20, wherein the ovarian cancer is a platinum-resistant ovarian cancer.

22. A kit comprising a first pharmaceutical composition comprising SBP-101 and a second pharmaceutical composition comprising doxorubicin.

23. A method of treating a patient having an ovarian cancer, the method comprising administering to the patient a combined effective amount of SBP-101 and an ornithine decarboxylase (ODC) inhibitor.

24. The method of claim 23, wherein the ornithine decarboxylase (ODC) inhibitor is DFMO.

25. The method of claim 23 or 24, wherein the ovarian cancer in the subject was previously treated with the anti-cancer agent or at least one other anti-cancer agent.

26. The method of any one of claims 23-25, wherein the ovarian cancer has developed resistance to a platinum-based therapy.

27. The method of any one of claims 23-26, wherein the ovarian cancer is a platinum-resistant ovarian cancer.

28. The method of any one of claims 23-27, further comprising administering at least one additional therapeutic to the subject.

29. The method of claim 28, wherein the at least one additional therapeutic is selected from the group consisting of surgical therapy, chemotherapy, radiation therapy, cryotherapy, hormonal therapy, toxin therapy, immunotherapy, or cytokine therapy.

30. The method of any one of claims 23-29, wherein the SBP-101 and the ODC inhibitor are administered consecutively.

31. The method of claim 30, wherein the SBP-101 is administered prior to the ODC inhibitor.

32. The method of claim 30, wherein the SBP-101 is administered after the ODC inhibitor.

33. The method of any one of claims 23-29, wherein the SBP-101 and the ODC inhibitor are administered simultaneously.

34. The method of claim 23 or 33, wherein the SBP-101 and the ODC inhibitor are co-formulated.

35. The method of any one of claims 23-34, wherein the patient is human.

36. A pharmaceutical composition comprising SBP-101, an ODC inhibitor, and a pharmaceutically acceptable carrier.

37. The pharmaceutical composition of claim 36, for use in the treatment of ovarian cancer.

38. A pharmaceutical composition comprising SBP-101, for use in the treatment of ovarian cancer in combination with a second pharmaceutical therapy comprising an ODC inhibitor.

39. A pharmaceutical composition comprising an ODC inhibitor, for use in the treatment of ovarian cancer in combination with a second pharmaceutical therapy comprising SBP-101.

40. The pharmaceutical composition for use of claim 38 or 39, wherein the ovarian cancer is a platinum-resistant ovarian cancer.

41. Use of SBP-101 in the manufacture of a medicament for the treatment of ovarian cancer in combination with a second medicament comprising an ODC inhibitor.

42. Use of an ODC inhibitor in the manufacture of a medicament for the treatment of ovarian cancer in combination with a second medicament comprising SBP-101.

43. Use of SBP-101 and an ODC inhibitor in the manufacture of a medicament for the treatment of ovarian cancer.

44. The use of any one of claims 41-43, wherein the ovarian cancer is a platinum-resistant ovarian cancer.

45. A kit comprising a first pharmaceutical composition comprising SBP-101 and a second pharmaceutical composition comprising an ODC inhibitor.
